(19) United States
(12) Patent Application Publication
(10) Pub. No.: US 2009/0025734 A1
(43) Pub. Date: Jan. 29, 2009

(54) Devices, systems, and methods to move or restrain the hyoid bone

(75) Inventors: Eric N. Doelling, Sunnyvale, CA (US); Edward M. Gillis, San Jose, CA (US); Ronald G. Lax, Port Orange, FL (US); Lionel M. Nelson, Los Altos Hills, CA (US); Joseph Paraschac, Campbell, CA (US)

Correspondence Address: MICHAEL W. HAAS;RESPIRONICS, INC., 1010 MURRY RIDGE LANE, MURRYSVILLE, PA, 15668, US

(73) Assignee: Koninklijke Philips Electronics N.V., Eindhoven (NL)

(21) Appl. No.: 12/072,680

(22) Filed: Feb. 27, 2008

Related U.S. Application Data
(60) Provisional application No. 60/903,741, filed on Feb. 27, 2007.

(51) Int. Cl. A61F 5/56; A61F 2/02

(52) U.S. Cl. 128/848; 623/11.11

(57) ABSTRACT

Systems and methods employ implants attached to the hyoid bone, thyroid cartilage, cricoid cartilage, or both the thyroid and cricoid cartilages and a source of magnetic force to move the hyoid bone, thyroid cartilage, cricoid cartilage, or both the thyroid and cricoid cartilages in the treatment of sleep disordered breathing, using attracting, repelling or a combination of attracting and repelling magnetic forces.

### RELATED APPLICATION

This application claims the benefit of U.S. provisional patent application Ser. No. 60/903,741 filed 27 Feb. 2007.

### FIELD OF THE INVENTION

The invention is directed to devices, systems, and methods for moving and/or restraining the hyoid bone, e.g., for the treatment of sleep-related breathing disorders such as snoring, upper airway resistance syndrome and obstructive sleep apnea.

### BACKGROUND OF THE INVENTION

I. Characteristics of Sleep Apnea

First described in 1965, sleep apnea is a breathing disorder characterized by brief interruptions (10 seconds or more) of breathing during sleep. Sleep apnea is a common but serious, potentially life-threatening condition, affecting as many as 18 million Americans.

There are two types of sleep apnea: central and obstructive. Central sleep apnea, which is relatively rare, occurs when the brain fails to send the appropriate signal to the breathing muscles to initiate respirations, e.g., as a result of brain stem injury or damage. Mechanical ventilation is the only treatment available to ensure continued breathing.

Obstructive sleep apnea (OSA) is far more common. Normally, the muscles of the upper part of the throat keep the airway open to permit air flow into the lungs. When the muscles of the soft palate, the base of the tongue, the pharyngeal walls, and the uvula (the small fleshy tissue hanging from the center of the back of the throat) relax and sag, the relaxed tissues may vibrate as air flows past the tissues during breathing, resulting in snoring. Snoring affects about half of men and 25 percent of women—most of whom are age 50 or older.

In more serious cases, the airway becomes blocked, making breathing labored and noisy, or even stopping it altogether. In a given night, the number of involuntary breathing pauses or “apneic events” may be as high as 20 to 30 or more per hour. These breathing pauses are almost always accompanied by snoring between apneic episodes, although not everyone who snores has the condition. Sleep apnea can also be characterized by choking sensations.

Lack of air intake into the lungs results in lower levels of oxygen and increased levels of carbon dioxide in the blood. The altered levels of oxygen and carbon dioxide alert the brain to resume breathing and cause arousal. The frequent interruptions of deep, restorative sleep often lead to early morning headaches, excessive daytime sleepiness, depression, irritability, and learning and memory difficulties.

The medical community has become aware of the increased incidence of heart attack, hypertension and stroke in people with moderate or severe obstructive sleep apnea. It is estimated that up to 50 percent of sleep apnea patients have high blood pressure.

During an apneic event, the sleeping person is unable to continue normal respiratory function and the level of oxygen saturation in the blood is reduced. The brain senses the condition and causes the sleeper to struggle and gasp for air. Breathing then resumes, often followed by continued apneic events. There are potentially damaging effects to the heart and blood vessels due to abrupt compensatory swings in blood pressure associated with apneic events. During each event, the sleeping person will be partially aroused from sleep, resulting in a greatly reduced quality of sleep and associated daytime fatigue.

Although low apneic events are normal in all persons and mammals, increased or excessive frequency of blockages is associated with more serious forms of the disease and opportunity for health damage. When the incidence of blockage is frequent, corrective action is often necessary and should be taken.

II. Sleep and the Anatomy of the Upper Airway

The upper airway consists of a conduit that begins at the nasal valve, situated in the tip of the nose, and extends to the larynx. Although all tissue along this conduit is dynamic and responsive to the respiratory cycle, only the pharynx (the portion that starts behind the nasal cavity and ends in its connections to the supraglottic larynx) is totally collapsible.

The cross sectional area of the upper airway varies with the phases of the respiratory cycle. At the initiation of inspiration (Phase I), the airway begins to dilate and then to remain relatively constant through the remainder of inspiration (Phase II). At the onset of expiration (Phase III) the airway begins to enlarge, reaching maximum diameter and then diminishing in size so that at the end of expiration (Phase IV), it is at its narrowest, corresponding to the time when the upper airway dilator muscles are least active, and positive intraluminal pressure is lowest. The upper airway, therefore, has the greatest potential for collapse and closure at end-expiration. [ref: Schwab R J, Goldberg A N. Upper airway assessment: radiographic and other imaging techniques. Otolaryngol Clin North Am 1998; 31:931-968]

Sleep is characterized by a reduction in upper airway dilator muscle activity. For the individual with obstructive sleep apnea (OSA) and perhaps the other disorders which comprise much of the group of entities called obstructive sleep-disordered breathing (SDB), it is believed that this change in muscle function causes pharyngeal narrowing and collapse. Two possible etiologies for this phenomenon in OSA patients have been theorized. One is that these individuals reduce the airway dilator muscle tone more than non-apneics during sleep (the neural theory). The other is that all individuals experience the same reduction in dilator activity in sleep, but that the apneic has a pharynx that is structurally less stable (the anatomic theory). Both theories may in fact be contributors to OSA, but current studies seem to support that OSA patients have an intrinsically structurally narrowed and more collapsible pharynx [ref: Isono S, Remmers J, Tanaka A Sho Y, Sato J, Nishino T. Anatomy of pharynx in patients with obstructive sleep apnea and in normal subjects. J Appl Physiol 1997:82:1319-1326.] Although this phenomenon is often accentuated at specific sites, such as the velopharyngeal level [Isono], studies of closing pressures [Isono] supports dynamic fast MRI imaging that shows narrowing and collapse usually occurs along the entire length of the pharynx [ref: Shellock F G, Schatz C J, Julien P, Silverman J M, Steinberg F, Foo T K F, Hopp M L, Westbrook P R. Occlusion and narrowing of the pharyngeal airway in obstructive sleep apnea: evaluation by ultrafast spoiled GRASS MR imaging. Am J of Roentgenology 1992:158:1019-1024].

III. Treatment Options

To date, the only modality that addresses collapse along the entire upper airway is through use of mechanical positive pressure breathing devices, such as continuous positive airway pressure (CPAP) machines. All other modalities, such as various surgical procedures and oral appliances, by their nature, address specific sectors of the airway (such as palate, tongue base and hyoid levels), but leave portions of pharyngeal wall untreated. This may account for the considerably higher success rate of CPAP over surgery and oral appliances in controlling OSA. Although CPAP, which in essence acts as an airway splint for the respiratory cycle, is highly successful, it has some very significant shortcomings. It can be cumbersome to wear and travel with, difficult to accept on a social level, and not tolerated by many (for reasons such as claustrophobia, facial and nasal mask pressure sores, airway irritation). These factors have lead to a relatively poor long-term compliance rate. One study has shown that 65% of patients abandon their CPAP treatment in 6 months.

An alternative method that “splints” the airway during sleep giving the benefits afforded by CPAP without some of its shortcomings would therefore be advantageous. In this method magnetic energy is used either attractively (opposite poles of two or more magnets facing one another, resulting in attractive forces) or repulsively (like poles of two or more magnets facing one another, resulting in forces which repel one another). Magnets implanted in the tongue interact either by attractive or repulsive forces with other magnets implanted in various organs of the upper airway system or external to the body within a neck collar.

Since the “splint” method using repelling magnetic forces does not eliminate all forms of magnetic interaction (i.e. torquing, decentering, and twisting forces), implants within the tongue and pharyngeal wall often are difficult to stabilize in their desired locations. The magnetic implants could interact with one another causing the implants to fold or lose their shape, as well as attract to magnetic instruments. Implants need to resist the tendency to rotate or migrate from their original implant position.

The need remains for simple, cost-effective devices, systems, and methods for treating sleep apnea.

### Summary of the Technical Features

The present invention provides devices, systems and methods comprising at least one ferromagnetic material sized and configured for attachment to a hyoid bone, thyroid cartilage, cricoid cartilage, or both the thyroid and cricoid cartilages and a source of magnetic force sized and configured for placement to interact with the ferromagnetic material to move the hyoid bone or any combination of the above-mentioned cartilages in at least one desired direction.

The invention is particularly useful to treat sleep disordered diseases such as Obstructive Sleep Apnea (OSA).

In one embodiment, the technical features provide an implant system comprising at least one ferromagnetic material sized and configured for attachment to a hyoid bone, and a source of magnetic force sized and configured for placement to interact with the ferromagnetic material to move or prevent or resist from moving the hyoid bone in at least one desired direction, or in at least two desired directions, or in an anterior, caudal, or cranial direction, or combinations thereof.

In one embodiment, the technical features provide an implant system comprising at least one ferromagnetic material sized and configured for attachment to a thyroid cartilage, cricoid cartilage, or both the thyroid and cricoid cartilages, and a source of magnetic force sized and configured for placement to interact with the ferromagnetic material to move or prevent or resist from moving the thyroid cartilage, cricoid cartilage, or both the thyroid and cricoid cartilages in at least one desired direction, or in at least two desired directions, or in an anterior, caudal, or cranial direction, or combinations thereof.

Any of the implant systems just described can include additional technical features, e.g., a source of magnetic force acting by attracting at least one ferromagnetic material, or a source of magnetic force acting by repelling the at least one ferromagnetic material, or the source of magnetic force acting by repelling at least two ferromagnetic materials on the lateral sides, while attracting at least one ferromagnetic material in the middle of the anatomical structure. For all of the above-mentioned technical features, the source of magnetic force is enclosed in a neck collar. The neck collar can include an interior space that allows tissue along the neck and the hyoid bone to move in response to magnetic forces.

In another embodiment, a method of treating sleep disordered breathing is provided using any of the implant systems just described.

Other technical features shall be apparent based upon the accompanying description, drawings, and claims.

### BRIEF DESCRIPTION OF THE DRAWINGS

FIG. 1 is an anatomic side view of the head and neck showing the location and orientation of the hyoid bone.

FIG. 2 is a right anterolateral anatomic view of a hyoid bone.

FIGS. 3A to 3D are anatomic views of the muscles attached to the hyoid bone.

FIGS. 4A to 4G are anatomic views of the larynx. FIG. 4A shows the larynx as a whole; FIG. 4B, the cartilages of the larynx; FIG. 4C, the ligaments of the larynx, anterolateral view; FIG. 4D, the muscular attachments to the larynx; FIG. 4E, the ligaments of the larynx, posterior view; FIG. 4F, the cricoid cartilage; and FIG. 4G, the muscles of the larynx when the right lamina of the thyroid cartilage is taken out.

FIGS. 5 and 6 are perspective anatomic views of implanted components of a system for moving or restraining a hyoid bone.

FIG. 7 is a perspective view of external components of the system, which interact with the implanted components shown in FIG. 5 or 6.

FIGS. 8A to 8F are anatomic side section views of the system of the implanted and external components shown in FIGS. 5, 6 and 7, in which the interaction between the components is a magnetic attracting force.

FIG. 9 is an anatomic top section view of the system of the implanted and external components in which the interaction between the components is a magnetic attracting force.

FIG. 10 is an anatomic top section view of the system of the implanted and external components shown in FIGS. 5, 6 and 7, in which the interaction between the components is a magnetic repelling force.

FIG. 11 is an anatomic top section view of the system of the implanted and external components shown in FIGS. 5, 6 and 7, in which the interaction between the components is a magnetic attracting force in the middle and a magnetic repelling force on both sides.

FIGS. 12A and 12B are anatomic side section views of the system of the implanted and external components shown in FIG. 11.

FIGS. 13, 14, and 15 are perspective anatomic views of implanted components of a system for moving or restraining a thyroid cartilage, cricoid cartilage, or both a thyroid and cricoid cartilages together.

FIGS. 16 and 17 are anatomic top section views of the system of the implanted and external components shown in FIGS. 13, 14, and 15.

### DESCRIPTION OF THE PREFERRED EMBODIMENTS

This Specification discloses various magnetic-based devices, systems, and methods for moving or restraining the hyoid bone and/or muscles attached to the hyoid bone. For example, the various aspects of the invention have application in procedures requiring the restriction of tissue collapse in and/or around the airway.

The devices, systems, and methods are particularly well suited for treating sleep disordered breathing, including sleep apnea. For this reason, the devices, systems, and methods will be described in this context. Still, it should be appreciated that the disclosed devices, systems, and methods are applicable for use in treating other dysfunctions elsewhere in the body, which are not necessarily sleep disorder related.

I. Anatomy of the Hyoid Bone, the Muscles Attached Thereto, the Thyroid and Cricoid Cartilages of the Larynx

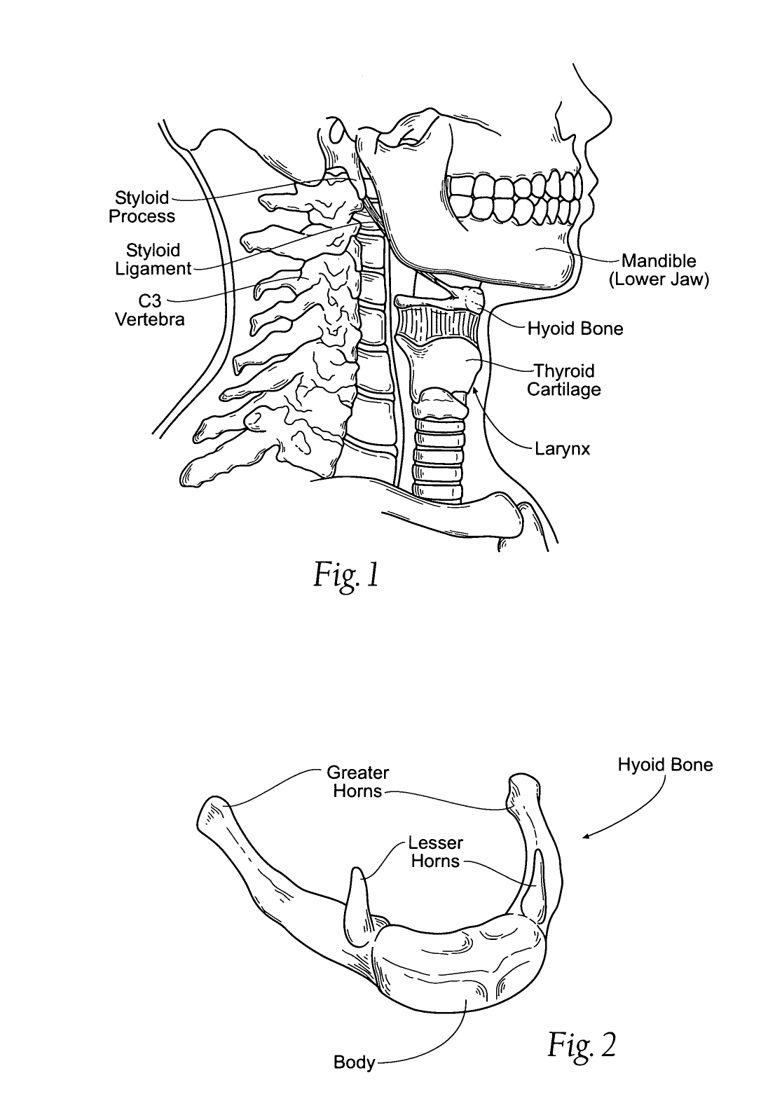

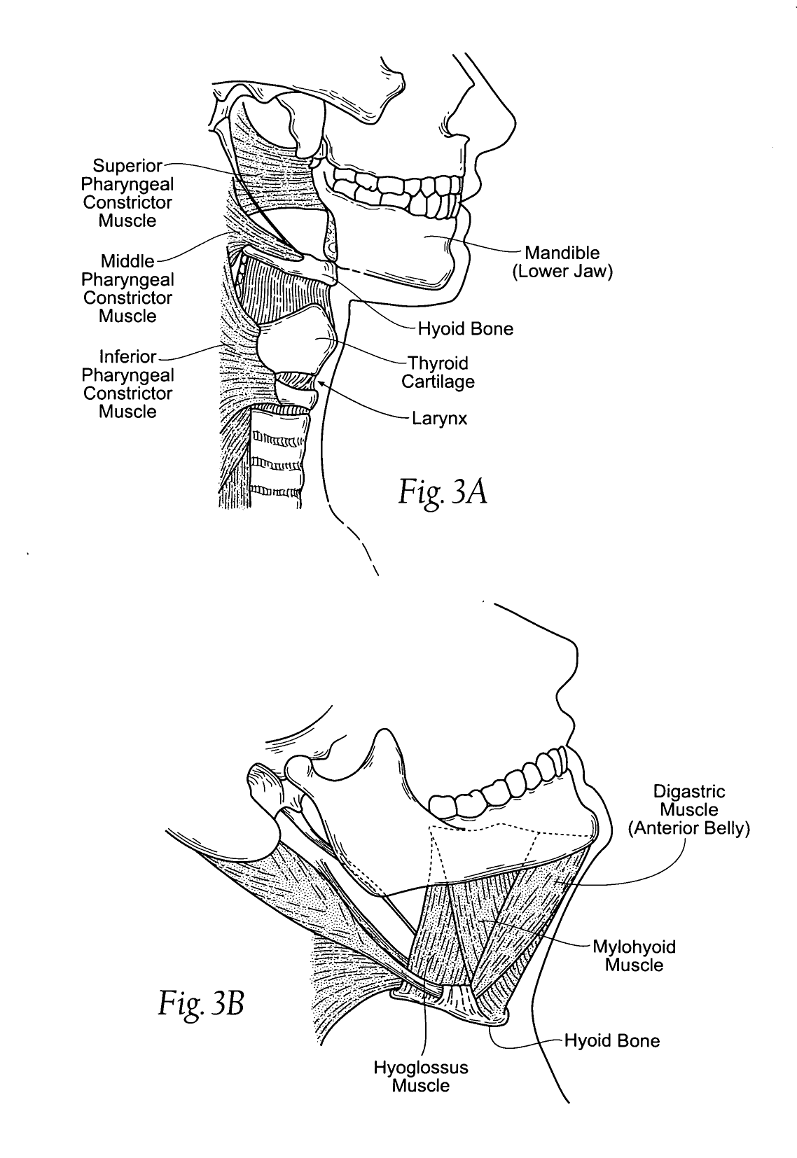

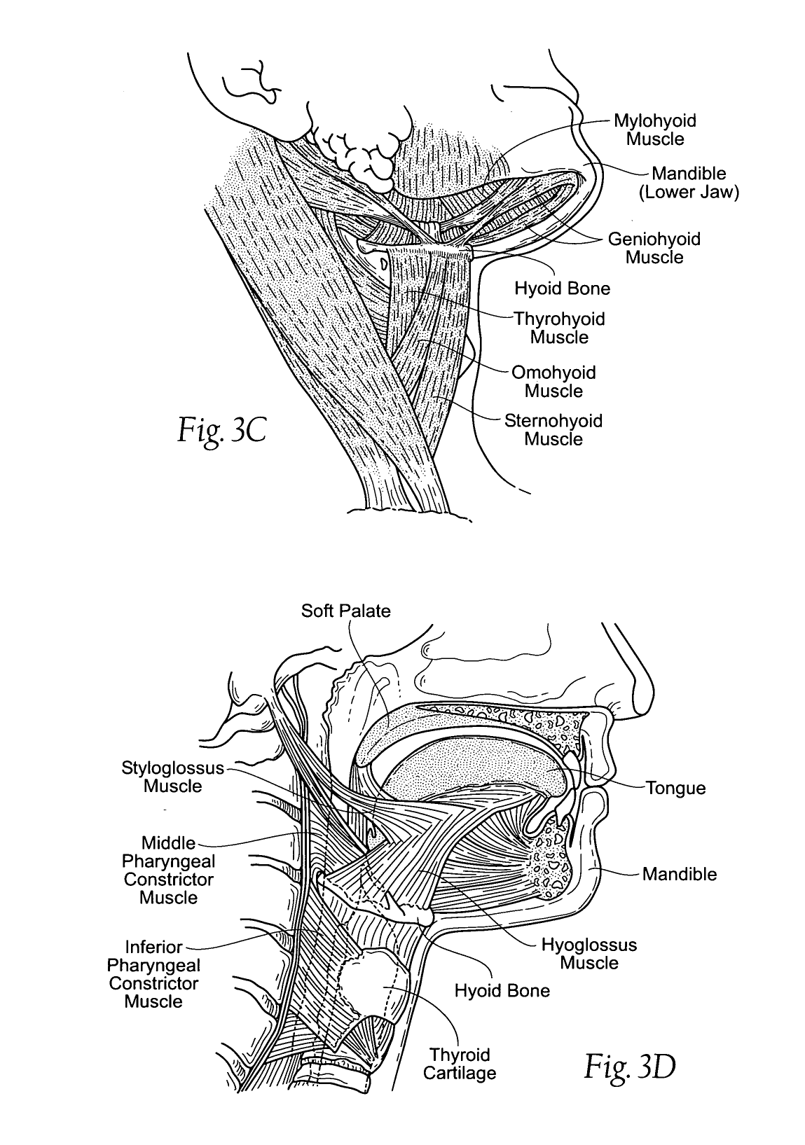

As FIG. 1 shows, the hyoid bone lies in the anterior part of the neck at the level of the C3 vertebra in the angle between the lower jaw (mandible) and the thyroid cartilage. It is a symmetric U-shaped bone (see FIG. 2), comprising a body with greater horns and lesser horns, which serve as points of attachment for numerous muscles in the tongue, pharynx, and the anterolateral part of the neck (see FIGS. 3A to 3D).

As best shown in FIG. 3D, the hyoid bone is situated at the root of the tongue in the front of the neck and between the lower jaw and the largest cartilage of the larynx, the voice box. The hyoid bone does not articulate with any other bone. It serves a purely anchoring function for muscles. The hyoid bone is suspended from the styloid processes of the temporal bones (see FIG. 1) by the stylohyoid ligaments and is firmly bound to the thyroid cartilage. Functionally, the hyoid bone serves as an attachment point for numerous muscles and a prop to keep the airway open. The primary function of the hyoid bone is to serve as an anchoring structure for the tongue.

FIGS. 3A to 3D show some of the numerous muscles that are attached to the hyoid bone. The muscles attached to the hyoid bone include the middle pharyngeal constrictor muscle (see FIG. 3A), which attaches at the end of the greater horns. The middle pharyngeal constrictor muscle, together with the superior and inferior pharyngeal constrictor muscles (also shown in FIG. 3A), extend along the upper airway. As before stated, a change in muscle function of the pharyngeal constrictor muscles can lead to pharyngeal narrowing and collapse.

The muscles attached to the hyoid bone also include the hyoglossus muscles (see FIGS. 3B and 3D). The hyoglossus muscles originate along the entire length of each greater horn and also from the body of the hyoid. The hyoglossus muscles are inserted into the posterior half or more of the sides of the tongue, as FIG. 3D best shows. The hyoid bone anchors the hyoglossus muscles when they contract, to depress the tongue and to widen the oral cavity, thereby opening the airway.

The muscles attached to the hyoid bone also include the two geniohyoid muscles (see FIG. 3C). The geniohyoid muscles originate close to the point at which the two halves of the lower jaw meet; the fibers of the muscles extend downward and backward, close to the central line, to be inserted into the body of the hyoid bone. Contraction of the geniohyoid muscles pulls the hyoid bone upward and forward, shortening the floor of the mouth and widening the pharynx.

Inserting into the middle part of the lower border of the hyoid bone are the sternohyoids (see FIG. 3C). The sternohyoids are long muscles arising from the breastbone and collarbone and running upward and toward each other in the neck. The sternohyoids depress the hyoid bone after it has been elevated during swallowing.

Other muscles attached to the hyoid bone are the two mylohyoid muscles (see FIG. 3C), which form a sort of diaphragm for the floor of the mouth, elevating the floor of the mouth and tongue during swallowing; the thyrohyoid (see FIG. 3C), arising from the thyroid cartilage of the larynx, which elevates the larynx; and the omohyoid (see FIG. 3C), which originates from the upper margin of the shoulder blade, which depresses, retracts, and steadies the hyoid bone.

The position of the hyoid bone with relation to the muscles attached to it has been likened to that of a ship steadied as it rides when anchored “fore and aft.” Through the muscle attachments, the hyoid plays an important role in mastication, in swallowing, and in voice production.

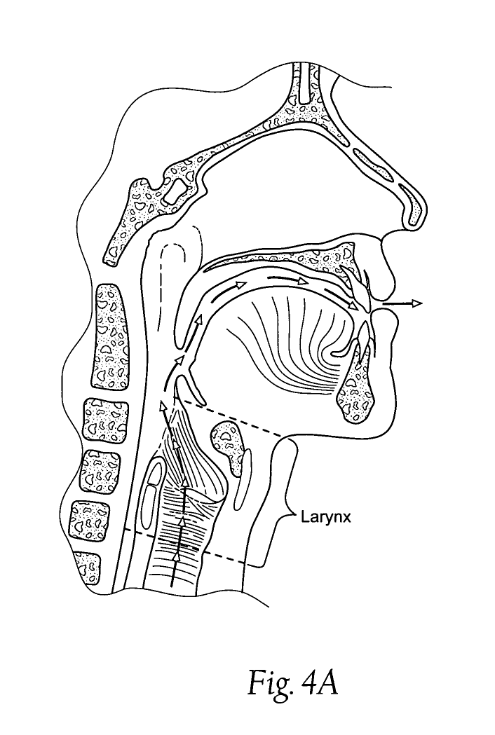

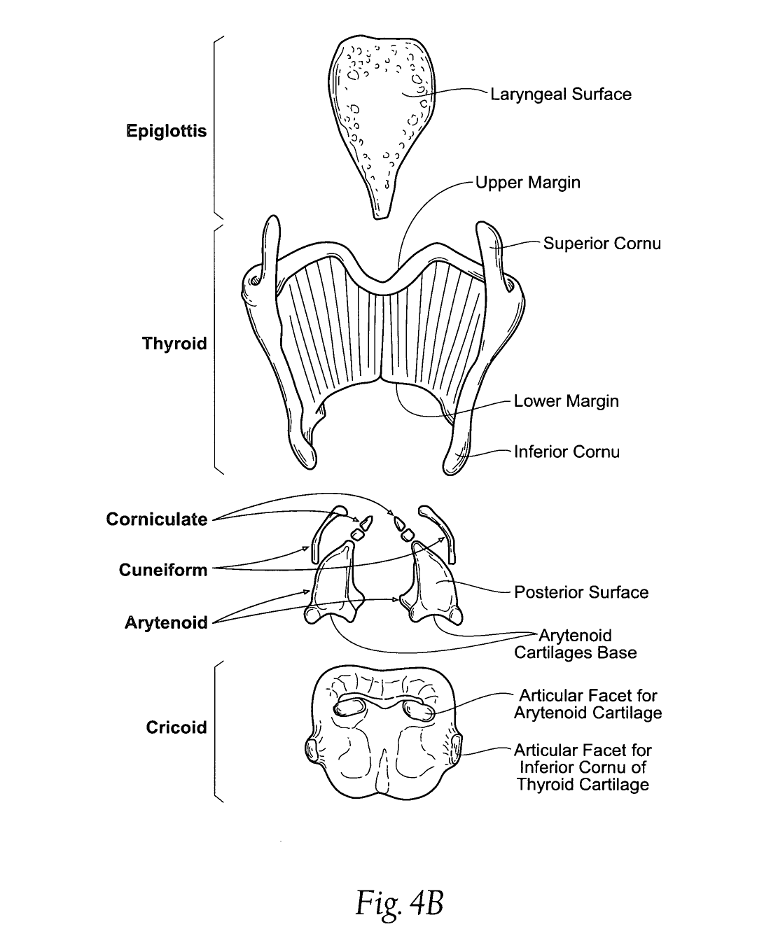

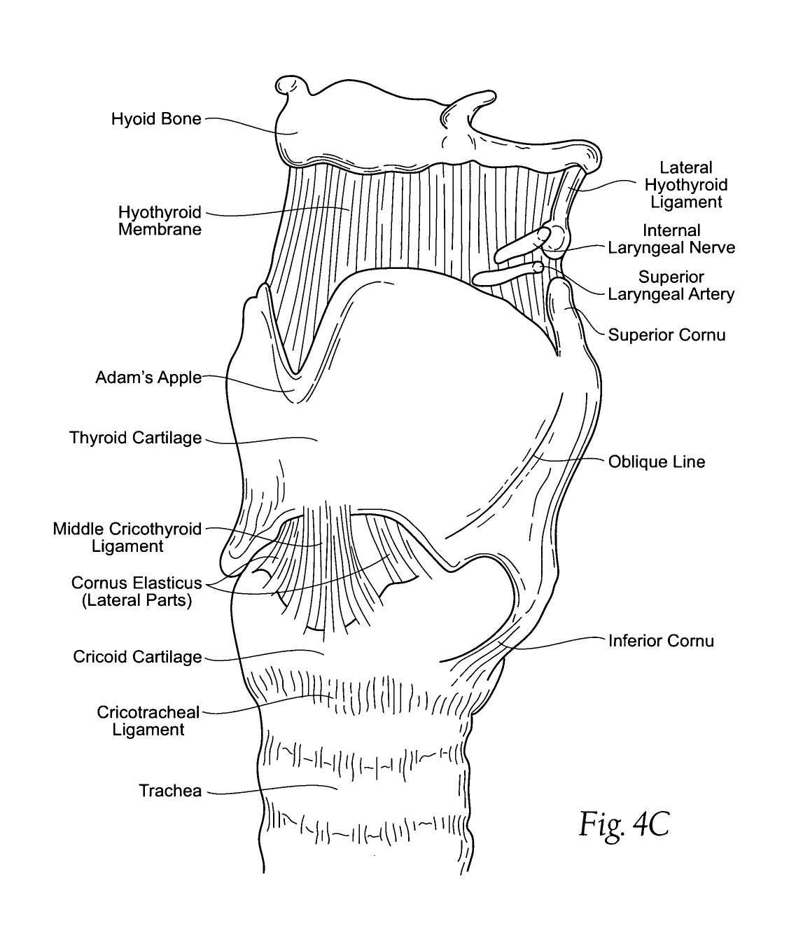

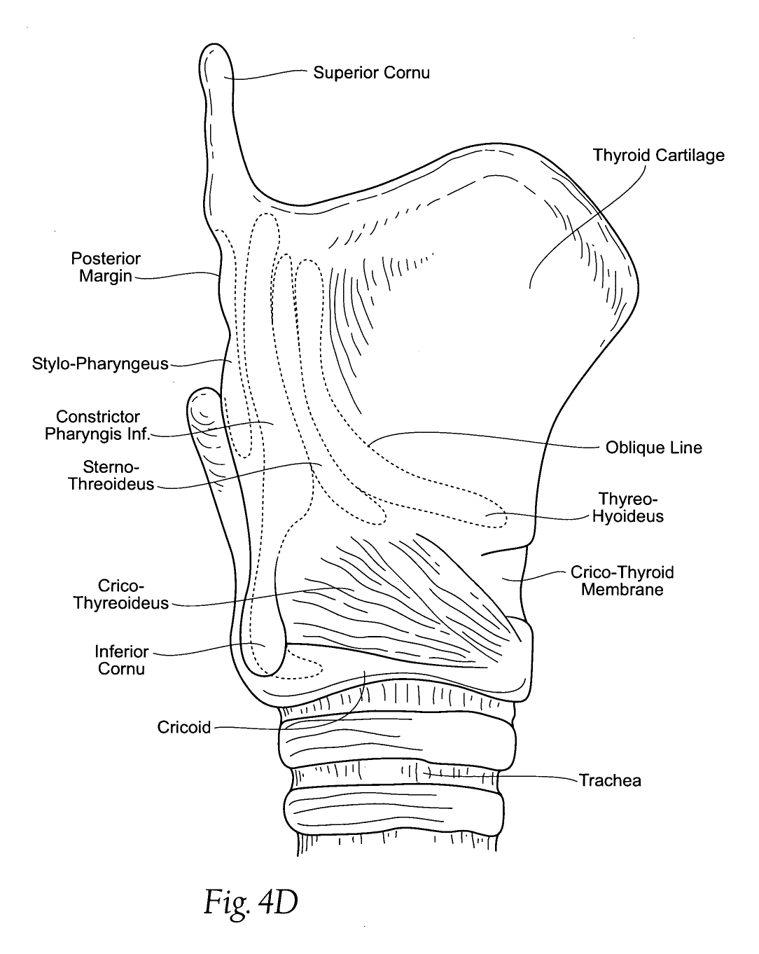

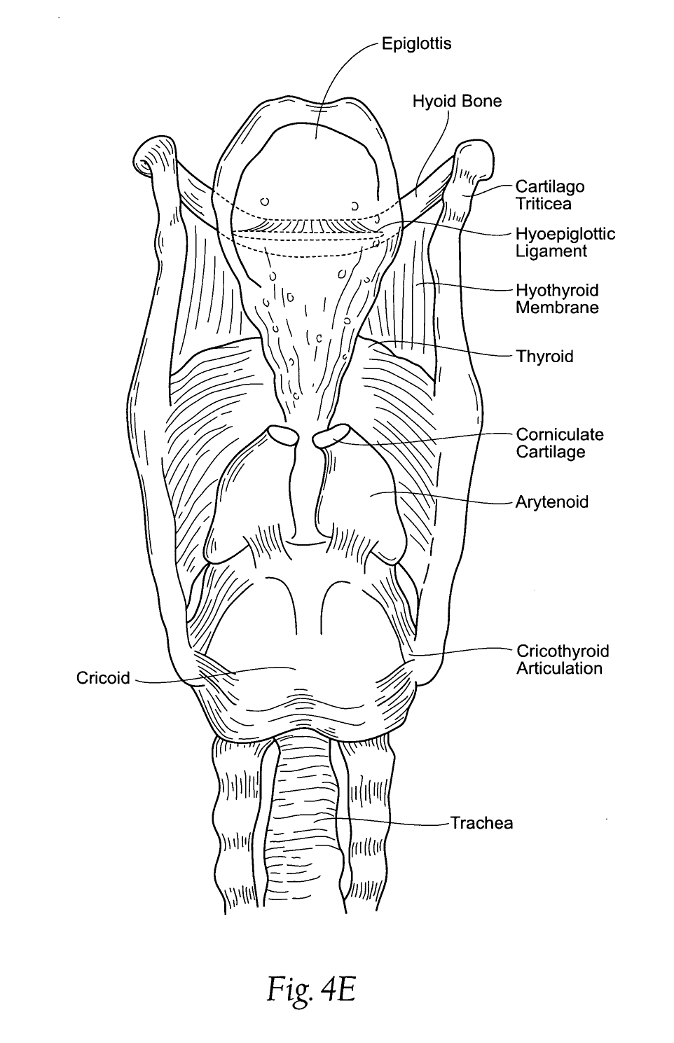

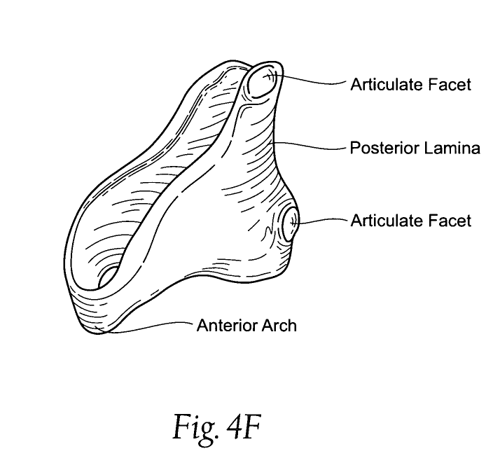

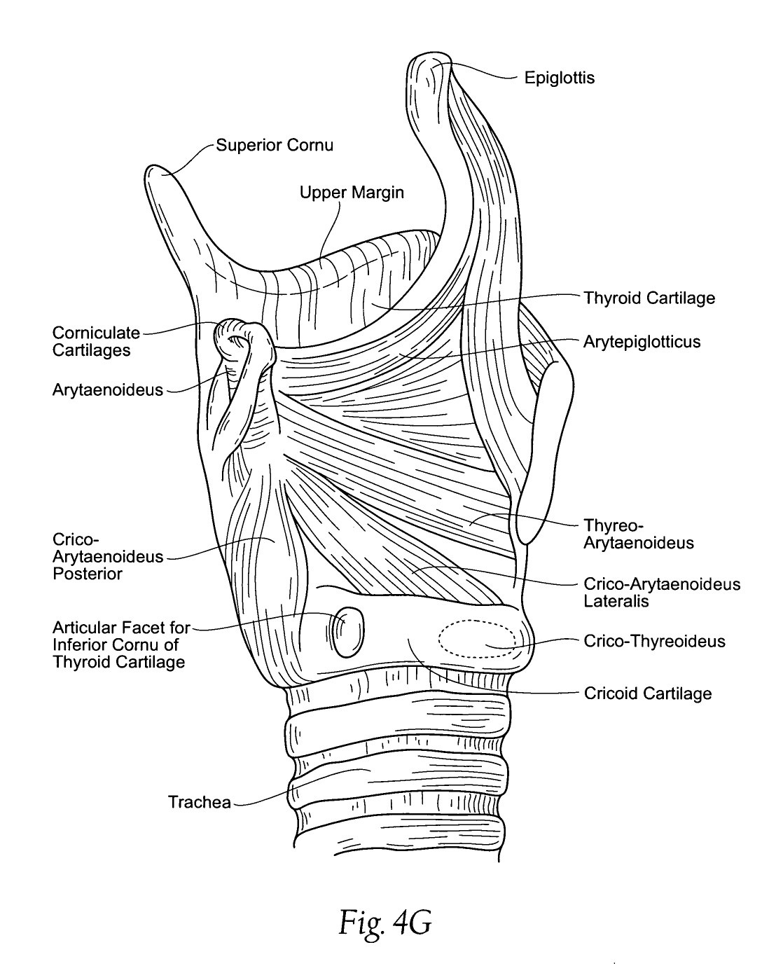

The larynx, also known as the organ of voice, is part of the upper respiratory tract. As FIG. 4A shows, the larynx is situated between the base of the tongue and the trachea; vertically, the larynx's position corresponds to the C4, C5, and C6 vertebrae, although this location is higher in females and during childhood. FIG. 4B shows the nine cartilages of the larynx: a thyroid, a cricoid, two arytenoids, two corniculate, two cuneiform, and an epiglottis.

Of the nine laryngeal cartilages, the thyroid cartilage is the largest in size (see FIG. 4C). The thyroid cartilage comprises two laminae whose anterior borders fuse at an acute angle, forming a projection called Adam's apple. Immediately above the Adam's apple is a V-shaped notch, the superior thyroid notch. The two laminae's posterior angles form projections or appendages called the superior and inferior cornua.

Along the outer surface of each lamina runs an oblique line, starting from the superior cornu and moving anterio-caudally towards the inferior thyroid tubercle on the lower margin of the thyroid cartilage (see FIG. 4D). The sternohyoid, thyrohyoid and inferior pharyngeal constrictor muscles are attached along this line.

The inner surface of each lamina is smooth and covered by a mucous membrane (see FIG. 4B). The angle formed by the fused laminae forms a point of attachment for the stem of the epiglottis, the ventricular and vocal ligaments, the thyroarytenoid, thyroepiglottic and vocal muscles, and the thyroepiglottic ligament.

The corresponding half of the hypothyroid membrane is attached along the upper margin of the lamina (see FIG. 4E). A small part of the lower margin near the middle is connected to the cricoid cartilage by the middle cricothyroid ligament.

The superior cornu is long and narrow, follows an upward, backward and medialward direction (see FIG. 4C). Its extremity attaches to the lateral hypothyroid ligament. The inferior cornu is short and thick, following a downward direction, slightly forward and medialward; its extremity articulates with the side of the cricoid cartilage.

Although smaller in size, the cricoid cartilage is both thicker and stronger than the thyroid, and constitutes the lower and posterior walls of the larynx (see FIG. 4F). The cricoid cartilage comprises a posterior quadrate lamina and a narrow anterior arch. The laminae are both wide and deep. Along the middle line of the cricoid cartilage extends a vertical ridge to the lower end where the longitudinal fibers of the esophagus connect; on each side of the vertical ridge lies a depression for the posterior cricoarytenoid muscle (see FIG. 4G).

The arch of the cricoid cartilage is thin and convex; the cricothyroid and inferior pharyngeal constrictor muscles are thereto attached (see FIGS. 4G and 4D). Small points of articulation with the inferior cornua of the thyroid cartilage cap the junction of the lamina and the arch on both sides (see FIG. 4C).

The lower border connects to the highest ring of the trachea via the cricotracheal ligament (see FIG. 4C). The upper border attaches, among others, to the middle of the cricothyroid ligament. The inner surface of the cricoid cartilage is smooth and lined with a mucous membrane.

The larynx comprises extrinsic ligaments which link the thyroid cartilage and the epiglottis with the hyoid bone and the cricoid cartilage with the trachea (see FIG. 4C). The hyothyroid membrane and the lateral hyothyroid ligament attach the thyroid cartilage to the hyoid bone. The hyoepiglottic ligament connects the epiglottis to the upper border of the hyoid bone. The cricotracheal ligament attaches the cricoid cartilage to the first ring of the trachea (see FIG. 4C).

The intrinsic ligaments of the larynx connect the various cartilages of the larynx to each other. Of particular interest is the conus elasticus (cricothyroid membrane) which connects the thyroid and cricoid cartilages. An articular capsule envelops the articulation of the inferior cornu of the thyroid with the cricoid cartilage on either side.

The articulation between the inferior cornu of the thyroid and cricoid cartilage on either side is diarthrodial, allowing free rotatory and gliding motion. The cricoid cartilage can rotate upon the inferior cornua of the thyroid cartilage around an axis that traverses both joints. The gliding movement comprises limited shifts of the crichoid on the thyroid in different directions.

The articulations between the arytenoid cartilages and the cricoid are also diarthrodial, encompassing two types of movement: 1. a rotatory movement of the arytenoid cartilages on a vertical axis (here the vocal process moves in a lateral and medial direction, and the rima glottides either grows or diminishes in size); and 2. a gliding movement (which permits the arytenoid cartilages to come closer or move farther away from each other). The articular surfaces' lateral gliding is accompanied by a forward and downward movement, when viewed from the direction and slope of the articular surfaces. The two movements of gliding and rotation are linked, the medial gliding joins with medialward rotation, and the lateral gliding with lateralward rotation. The posterior cricoarytenoid ligaments restrain the anterior movement of the arytenoid cartilages on the cricoid.

II. Systems and Methods for Moving or Restraining the Hyoid Bone, Thyroid Cartilage and Cricoid Cartilage

The middle pharyngeal constrictor muscle, hyoglossus muscle and geniohyoid muscle are all attached to the hyoid bone. Further attached to or affected by these muscles are the genioglossus and styloglossus muscles, thyrohyoid muscle, superior pharyngeal constrictor muscle and the inferior pharyngeal constrictor muscle. Each of these muscles affects the “openness” of pharyngeal airway, to a greater or lesser extent. Through manipulation of the hyoid bone, the pharynx can be caused to remain patent (open) [ref Moore K L, Dalley A F. Clinically Oriented Anatomy, Fifth Ed. Lippincott Williams & Wilkins 2006: 1047-1049], even with events that would normally result in a hypopneic or apneic event.

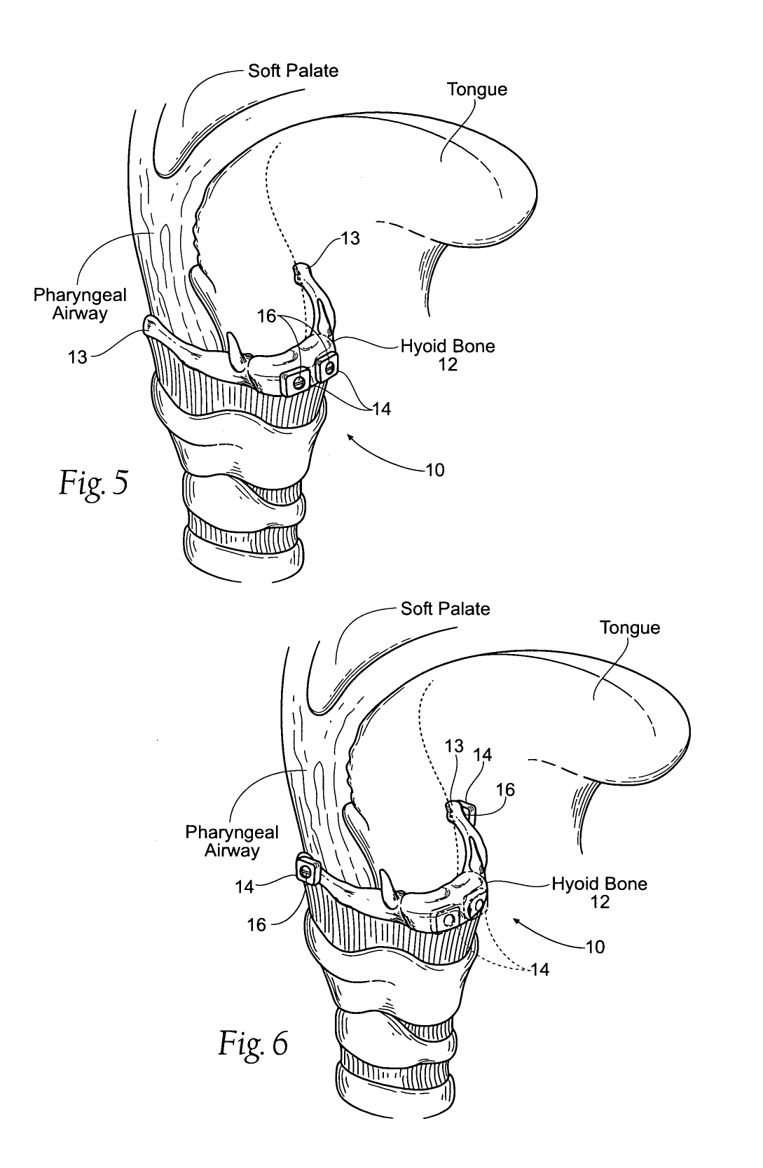

FIG. 5 shows representative implanted components of a system 10 for moving or restraining a hyoid bone 12. FIG. 6 shows representative external components of the system 10, which interact with the implanted components shown in FIG. 5.

As FIG. 5 shows, the system 10 includes one or more implanted magnetic or ferrous structures 14 that are affixed by an attachment means 16 to the hyoid bone 12. The attachment means 16 may take the form, fit, and function of a bone screw, or a metallic clamp ring or staple, or stainless wire, or other suitable mechanical fixation means using medically proven and accepted materials and methods. The attachment means 16 may also take the form of an adhesive, used alone or in combination with mechanical fixation. Bony in-growth can also be induced to augment the fixation.

One or more magnets or ferrous structures 14 may be affixed to the body of the hyoid bone 12, as FIG. 5 shows. Alternatively, or in combination (as FIG. 6 shows), one or more magnets or ferrous structures 14 can be affixed by attachment means 16 to one or more of the greater horn(s) 13 of the hyoid bone 12.

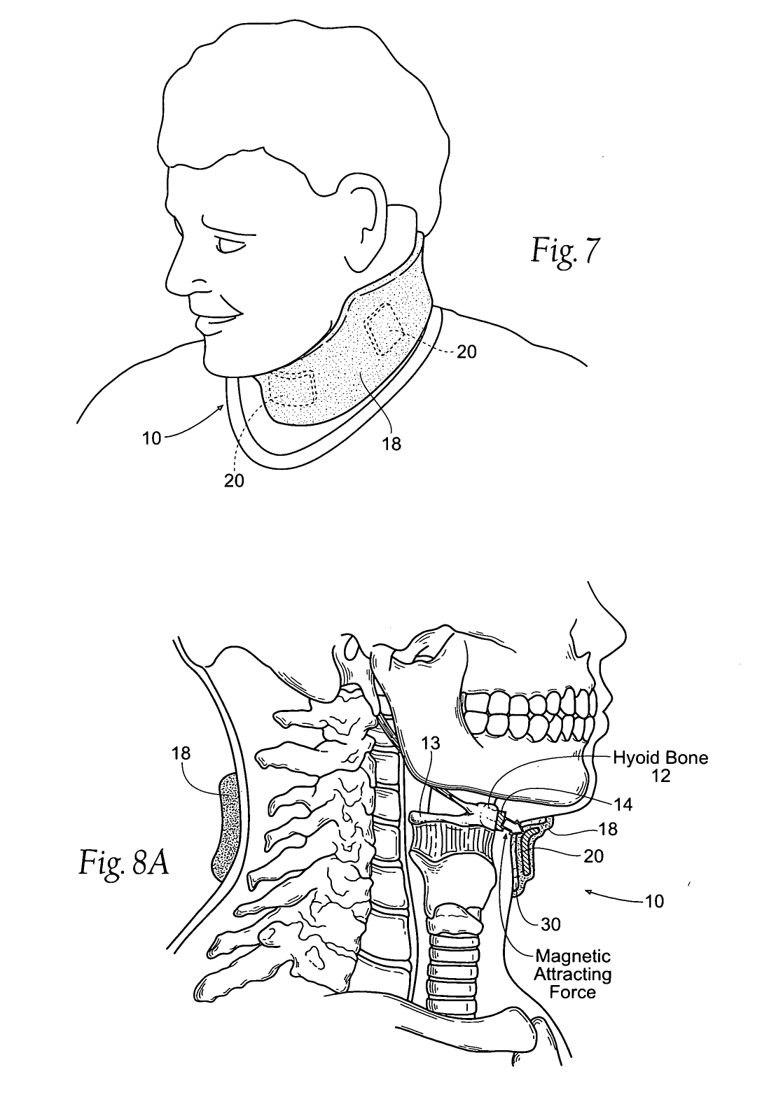

Following surgical implantation and attachment of the magnetic structures, a suitable time period may be allowed to pass for healing of the surgical incision and reduction in swelling and soreness. After that period, an external collar 18 (see FIG. 7) may be fitted by the physician.

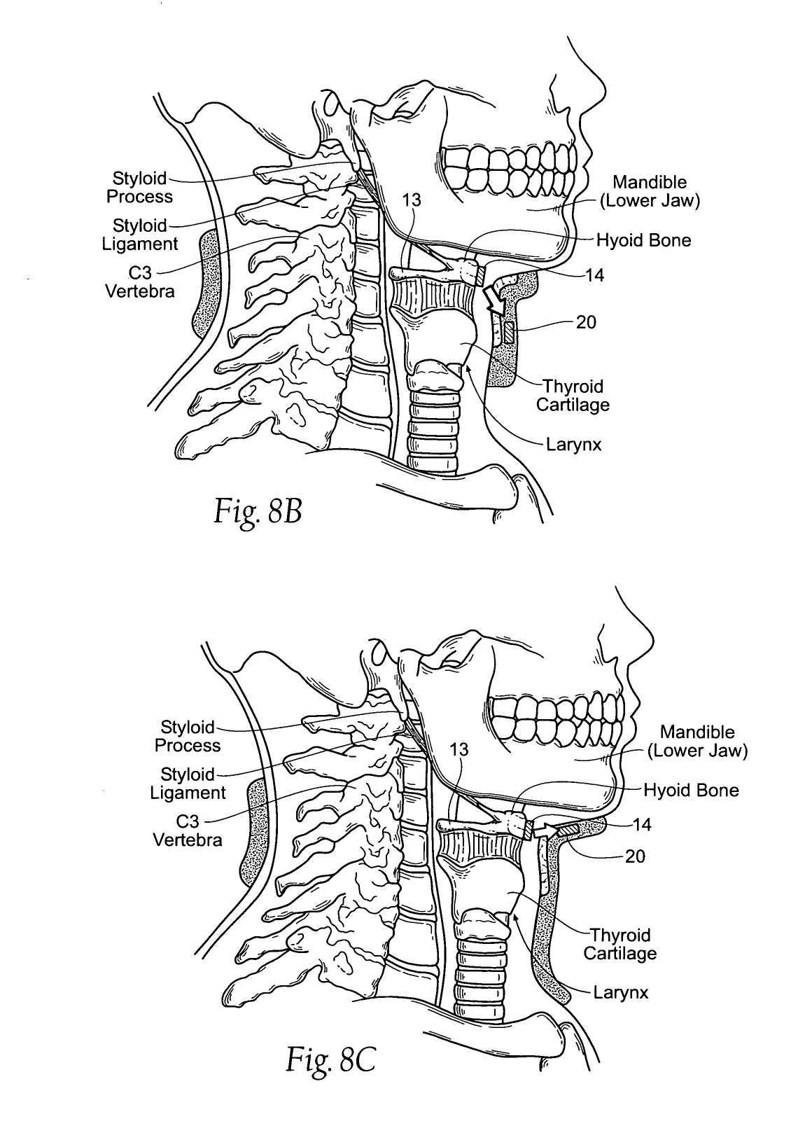

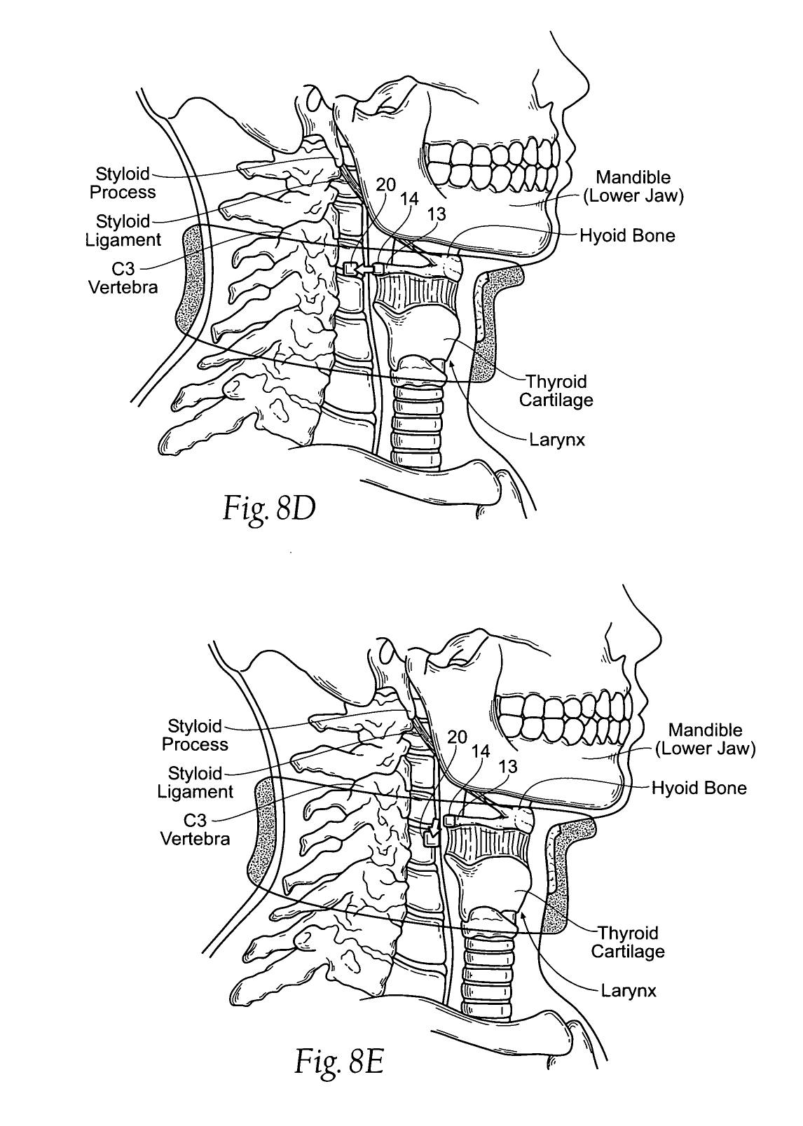

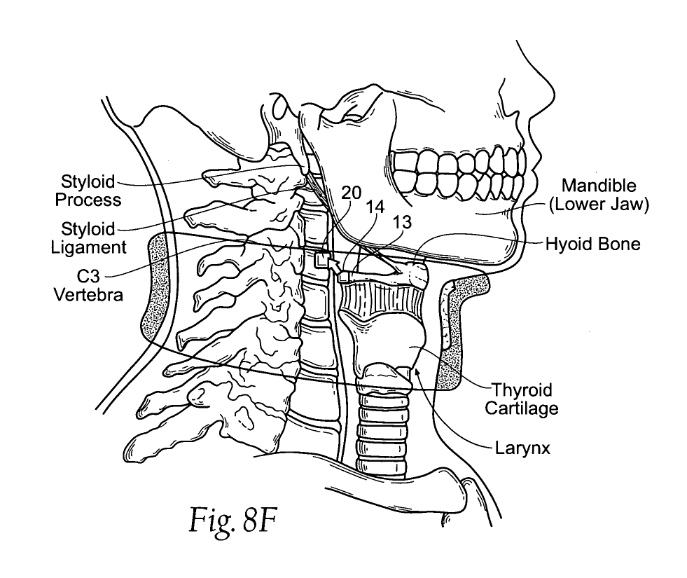

The collar 18 holds one or more external magnets 20. The vertical position of the magnets in the external collar will depend on the type of movement expected from the hyoid bone. For example, if the hyoid bone needs to move anteriorly, then the collar-borne magnets will be positioned at the level and opposing the position of the implanted magnetic or ferromagnetic structures 14 (see FIG. 8A). The polarity of the magnets 20 is selected to magnetically interact with the implanted magnetic or ferrous structures 14 by magnetic attraction.

The external collar 18 with magnets 20 will thereby attract the implanted magnetic or ferrous structures 14. The magnetic force of attraction will move the hyoid bone 12 toward the collar-borne magnets 20. The various muscles attached to or affected by the position and shape of the hyoid bone 12 can all affect airway patency and the system 10 is therefore utilized for treatment of the sleep related disorders.

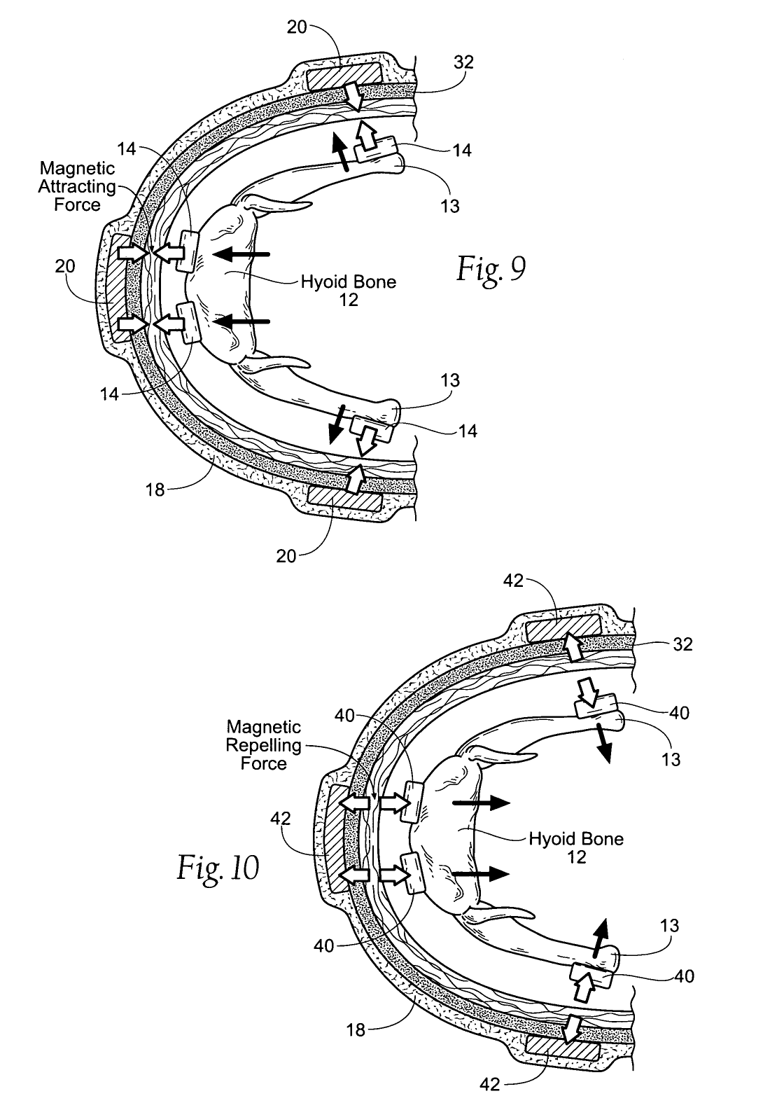

The force of attraction acting upon a magnetic or ferrous structure 14 affixed to the body of the hyoid bone 12 will cause an anterior displacement of the hyoid bone 12 toward an opposing collar-borne magnet 20 (see FIG. 9). As seen in FIG. 8D, the force of attraction acting upon a magnetic or ferrous structure 14 affixed to a greater horn of the hyoid bone 12 will cause a lateral displacement of the hyoid bone 12 toward an opposing collar-borne magnet 20. The anterior and lateral attractions will occur simultaneously, depending upon the orientation and opposition of the implanted structures 14 and collar-borne magnets 20.

If the physician desires caudal movement of the hyoid bone 12, the physician affixes one or more magnetic or ferrous structures 14 to the body of the hyoid bone, as seen in FIG. 8B. In this arrangement, the magnetic collar 18 is worn with one or more magnets 20 arranged opposite but below the level of the body of the hyoid bone, to relocate or attract the hyoid bone in a caudal direction.

If the physician desires caudal movement of the greater horns 13 of the hyoid bone 12, the physician affixes one or more magnetic or ferrous structures 14 to one or more of the greater horns of the hyoid bone, as seen in FIG. 8E. In this arrangement, the magnetic collar 18 is worn with one or more magnets 20 arranged opposite, but below the respective greater horn or horns of the hyoid bone, to relocate or attract the hyoid bone in a caudal direction.

Similarly, if the physician desires cranial movement of the hyoid bone 12, the physician affixes one or more magnetic or ferrous structures 14 to the body of the hyoid bone, as seen in FIG. 8C. In this arrangement, the magnetic collar 18 is worn with one or more magnets 20 arranged opposite but above the level of the body of the hyoid bone, to relocate or attract the hyoid bone in a cranial direction.

If the physician desires cranial movement of the greater horns 13 of the hyoid bone 12, the physician affixes one or more magnetic or ferrous structures 14 to one or more of the greater horns of the hyoid bone, as seen in FIG. 8F. In this arrangement, the magnetic collar 18 is worn with one or more magnets 20 arranged opposite, but above the respective greater horn or horns of the hyoid bone, to relocate or attract the hyoid bone in a cranial direction.

Depending on the physician's diagnosis, any combination of anterior, lateral, caudal, or anterior cranial displacement of the hyoid bone as a whole and/or the greater horns of the hyoid bone may be selected for treatment, depending upon each individual patient's needs.

Desirably, the collar 18 is sized and configured to form a space 30 (see FIGS. 8A-8F) between the interior of the collar and the external surface of the neck. This space 30 allows the external surface of the neck and the hyoid bone to move forward when the magnet or magnets 20 in the collar 18 exert an attractive force on the hyoid bone magnetic implant or implants. Desirably, the space 30 in the collar 18 is adjustable for more or less hyoid movement. The hyoid implant will move to a positive stop each time.

Desirably, the collar 18 is sized and configured to include a protective spacing pad 32 proximal to the magnet 20 (as FIG. 9 shows). The spacing pad 32 is desirably soft for patient comfort but serves another more important function. Larger and therefore stronger magnets 20 can exert large attractive forces that increase exponentially with reduced distance between the magnetic bodies. Tissue damage can result from very high attractive forces when the two complementary magnets (or a single magnet and a ferrous structure) come very close to one another. The spacing pad 32 prevents movement into this close proximity and avoids tissue damage.

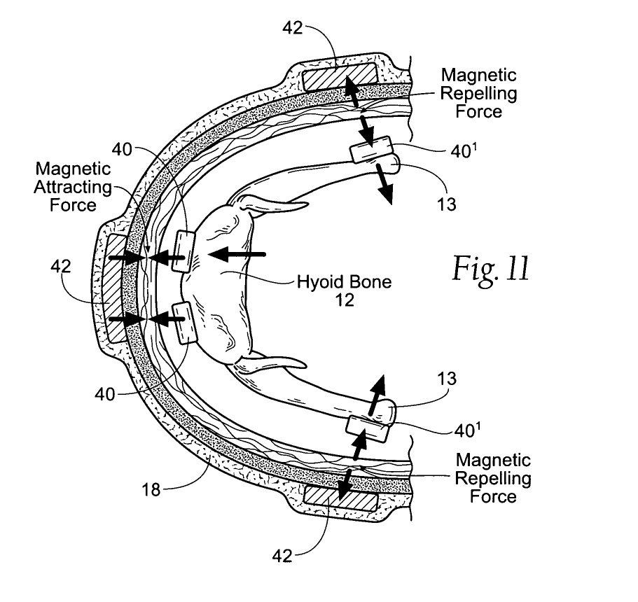

The direction of displacement of the body of the hyoid bone 12 or the greater horns of the hyoid bone 12 may in some cases be opposite to that described above. In this event (see FIG. 10), the poles of implanted magnets 40 and external collar-borne magnets 42 may be arranged in a like orientation, to provide a repelling force. This will cause the implanted magnets 40 and the hyoid bone 12 (or portions of the hyoid bone) to be displaced either posteriorly in the case of the body mounted magnet or laterally inward in the case of magnets affixed to the greater horn of the hyoid bone. This condition might occur when the constrictor muscles at the back of the airway have been determined to be the major factor in the sleep related breathing disorder. Then, anterior or inward displacement of the hyoid bone may cause lessening of the constriction on the airway and relief to the patient.

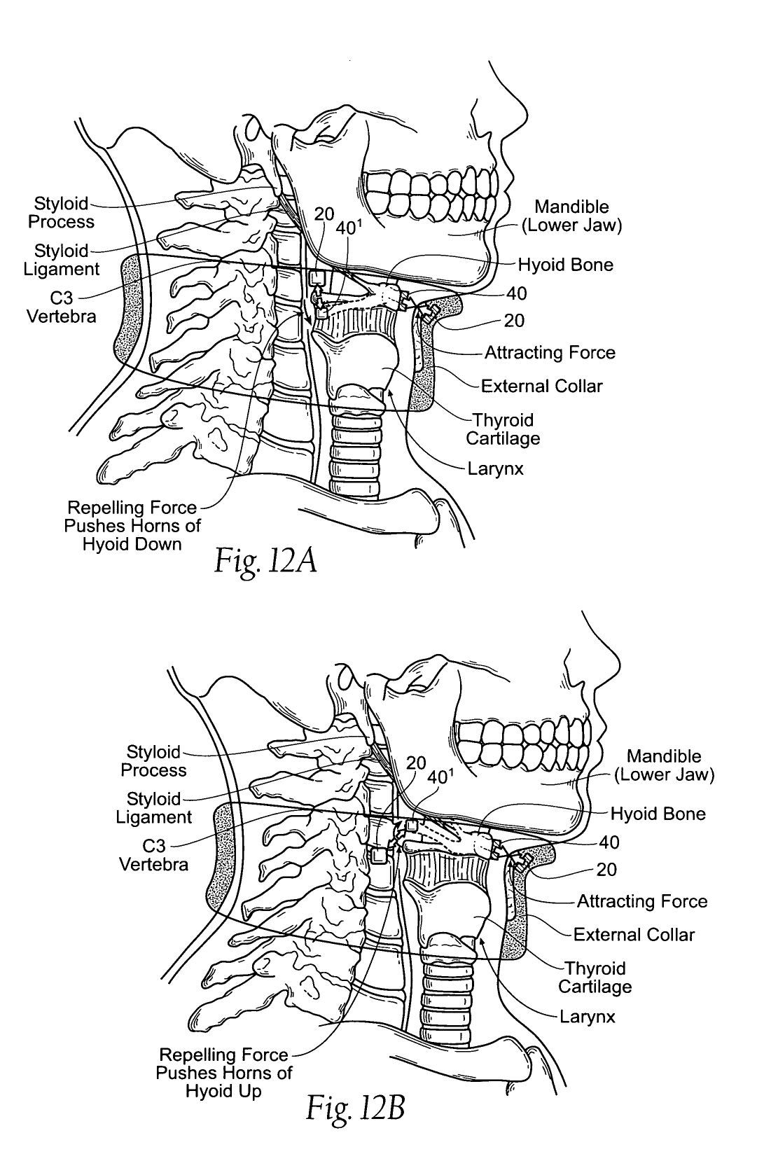

In yet another alternative embodiment (see FIG. 11), the poles of implanted magnets 40 and 40′ will be opposite so that when interacting with the external collar-borne magnets 42, implanted magnets 40 will be attracted, while implanted magnets 40′ will be repelled. These interactions will cause the hyoid bone as a whole to move anteriorly, while the greater horns of the hyoid will move either caudally, cranially, or laterally inward depending upon the location of the magnets on the external collar (see FIGS. 12A and 12B, respectively). An individual patient's therapeutic needs will determine whether a caudal, cranial, or laterally inward movement of the greater horns of the hyoid bone should be used.

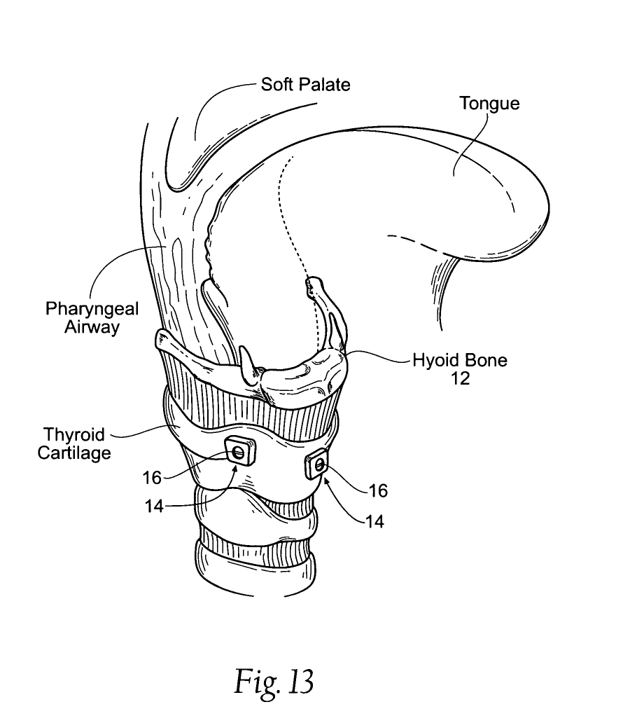

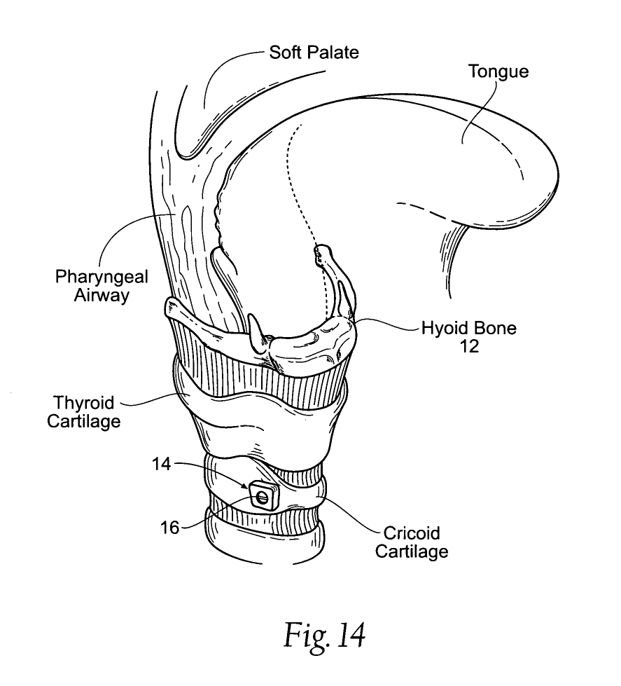

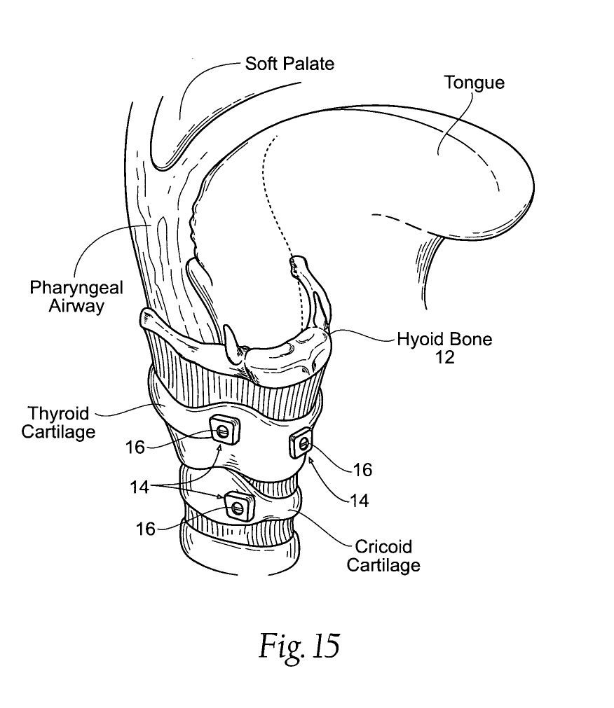

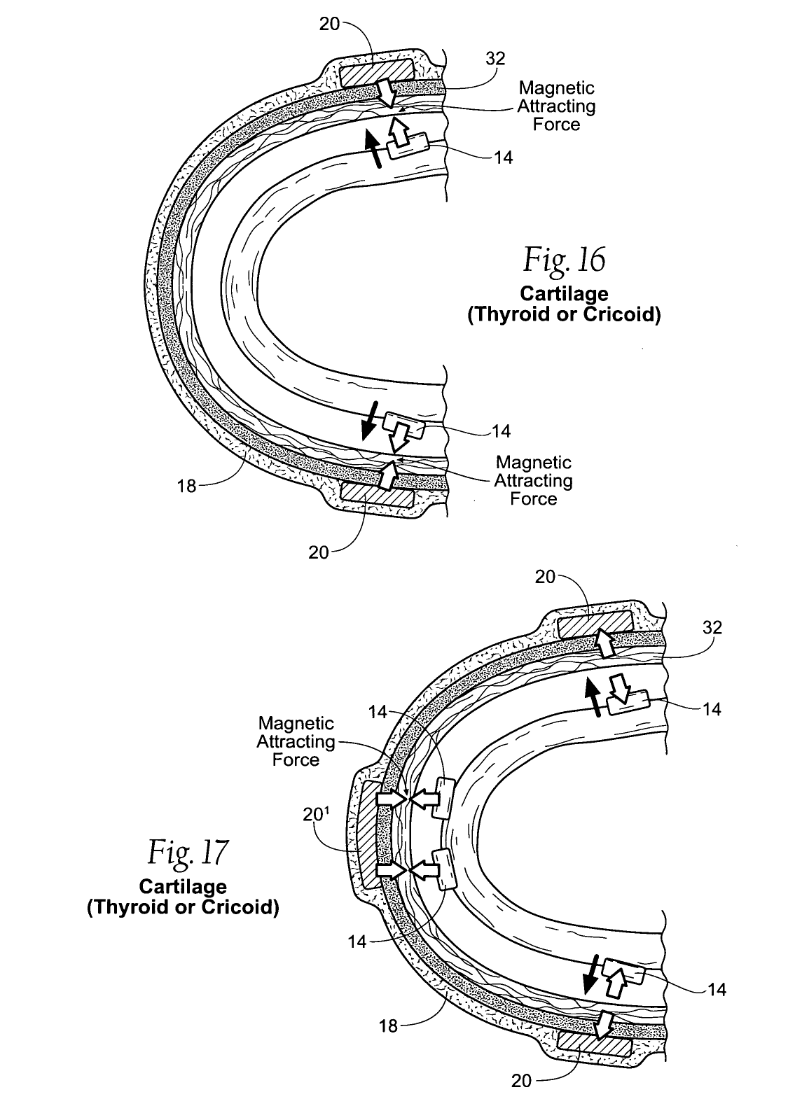

In an alternative embodiment, one or more magnets or ferrous structures 14 can be affixed by attachment means 16 to the thyroid cartilage, the crichoid cartilage, or both the thyroid and crichoid cartilages, as shown in FIGS. 13, 14, and 15. FIGS. 16 and 17 show the interaction between the magnetic poles of the magnets attached to the thyroid and/or crichoid cartilage(s) and the collar-anchored magnets 20′. Both types of magnets are angled so that the attracting force between the magnets attached to either the thyroid cartilage, crichoid cartilage or both the thyroid and crichoid cartilages and the collar-anchored magnets 20′, pulls the corresponding cartilage(s) caudally, thereby causing the airway to become patent and prevent apneic events.

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the technical features of the invention.

We claim:

1. An implant system comprising
at least one ferromagnetic material sized and configured for attachment to a hyoid bone, and
a source of magnetic force sized and configured for placement to interact with the ferromagnetic material to move the hyoid bone in at least one desired direction.

2. An implant system comprising
at least one ferromagnetic material sized and configured for attachment to a hyoid bone, and
a source of magnetic force sized and configured for placement to interact with the ferromagnetic material to move the hyoid bone in at least two desired directions.

3. An implant system comprising
at least one ferromagnetic material sized and configured for attachment to a hyoid bone, and
a source of magnetic force sized and configured for placement to interact with the ferromagnetic material to move the hyoid bone in an anterior direction.

4. An implant system comprising
at least one ferromagnetic material sized and configured for attachment to a hyoid bone, and
a source of magnetic force sized and configured for placement to interact with the ferromagnetic material to move the hyoid bone in a posterior direction.

5. An implant system comprising
at least one ferromagnetic material sized and configured for attachment to a hyoid bone, and
a source of magnetic force sized and configured for placement to interact with the ferromagnetic material to move the hyoid bone in a lateral direction.

6. An implant system according to claim 1 or 2
wherein the source of magnetic force acts by attracting the at least one ferromagnetic material.

7. An implant system according to claim 1 or 2
wherein the source of magnetic force acts by repelling the at least one ferromagnetic material.

8. An implant system according to claims 1 or 2
further comprising at least two ferromagnetic materials sized and configured for attachment to a hyoid bone, and
wherein the source of magnetic force acts by attracting at least one ferromagnetic material and repelling at least one other ferromagnetic metal.

9. An implant system according to claims 1 or 2
wherein the source of magnetic force is enclosed in a neck collar.

10. An implant system according to claims 1 or 2
wherein the neck collar has an interior collar and includes space in the interior collar to allow the surface of the neck and hyoid bone to move anteriorly.

11. A method comprising:
providing at least one ferromagnetic material sized and configured for attachment to a hyoid bone;
providing a source of magnetic force sized and configured for placement to interact with the at least one ferromagnetic material; and
moving the hyoid bone in at least one direction due to interaction between the at least one ferromagnetic material and the source of magnetic force.

12. The method according to claim 11
wherein the moving of the hyoid bone treats sleep disordered breathing.

13. An implant system comprising
at least one ferromagnetic material sized and configured for attachment to a thyroid cartilage, and
a source of magnetic force sized and configured for placement to interact with the ferromagnetic material to move the thyroid cartilage in at least one desired direction.

14. An implant system comprising
at least one ferromagnetic material sized and configured for attachment to a thyroid cartilage, and
a source of magnetic force sized and configured for placement to interact with the ferromagnetic material to move the thyroid cartilage in a caudal direction.

15. An implant system according to claim 13
wherein the source of magnetic force acts by attracting the at least one ferromagnetic material.

16. An implant system according to claim 13
wherein the source of magnetic force is enclosed in a neck collar.

17. An implant system according to claim 16
wherein the neck collar has an interior collar and includes space in the interior collar to allow the surface of the neck and thyroid cartilage to move caudally.

18. A method comprising
providing at least one ferromagnetic material sized and configured for attachment to the thyroid cartilage;
providing a source of magnetic force sized and configured for placement to interact with the at least one ferromagnetic material; and
moving the thyroid cartilage in at least one direction due to interaction between the at least one ferromagnetic material and the source of magnetic force.

19. The method according to claim 18
wherein the moving of the thyroid cartilage treats sleep disordered breathing.

20. An implant system comprising
at least one ferromagnetic material sized and configured for attachment to a cricoid cartilage, and
a source of magnetic force sized and configured for placement to interact with the ferromagnetic material to move the cricoid cartilage in at least one desired direction.

21. An implant system comprising
at least one ferromagnetic material sized and configured for attachment to a cricoid cartilage, and
a source of magnetic force sized and configured for placement to interact with the ferromagnetic material to move the cricoid cartilage in a caudal direction.

22. An implant system according to claim 20
wherein the source of magnetic force acts by attracting the at least one ferromagnetic material.

23. An implant system according to claim 20
wherein the source of magnetic force is enclosed in a neck collar.

24. An implant system according to claim 23 wherein the neck collar has an interior collar and includes space in the interior collar to allow the surface of the neck and cricoid cartilage to move caudally.

25. A method comprising
providing at least one ferromagnetic material sized and configured for attachment to the cricoid cartilage;
providing a source of magnetic force sized and configured for placement to interact with the at least one ferromagnetic material; and
moving the cricoid cartilage in at least one direction due to interaction between the at least one ferromagnetic material and the source of magnetic force.

26. The method according to claim 25
wherein the moving of the cricoid cartilage treats sleep disordered breathing.

27. An implant system comprising
at least one ferromagnetic material sized and configured for attachment to thyroid and cricoid cartilages, and
a source of magnetic force sized and configured for placement to interact with the ferromagnetic material to move the thyroid and cricoid cartilages in at least one desired direction.

28. An implant system comprising
at least one ferromagnetic material sized and configured for attachment to a thyroid and cricoid cartilages, and
a source of magnetic force sized and configured for placement to interact with the ferromagnetic material to move the thyroid and cricoid cartilages in a caudal direction.

29. An implant system according to claim 27
wherein the source of magnetic force acts by attracting the at least one ferromagnetic material.

30. An implant system according to claim 27
wherein the source of magnetic force is enclosed in a neck collar.

31. An implant system according to claim 30
wherein the neck collar includes space in the interior collar to allow the surface of the neck and thyroid and cricoid cartilages to move caudally.

32. A method comprising
providing at least one ferromagnetic material sized and configured for attachment to the thyroid and cricoid cartilages;
providing a source of magnetic force sized and configured for placement to interact with the at least one ferromagnetic material; and
moving the thyroid and cricoid cartilages in at least one direction due to interaction between the at least one ferromagnetic material and the source of magnetic force.

33. The method according to claim 32
wherein the moving of the thyroid and cricoid cartilages treats sleep disordered breathing.
